(19) United States
(12) Patent Application Publication
(10) Pub. No.: US 2006/0135599 A1
(43) Pub. Date: Jun. 22, 2006

(54) Use for pharmaceutical composition

(75) Inventors: Catherine Symonds, Salisbury (GB); Jonathan Berman, Stanmore (GB)

Correspondence Address: WOLF GREENFIELD & SACKS, PC;FEDERAL RESERVE PLAZA, 600 ATLANTIC AVENUE, BOSTON, MA, 02210-2206, US

(73) Assignee: GW Pharma Limited, Salisbury (GB)

(21) Appl. No.: 10/533,504

(22) PCT Filed: Nov. 3, 2003
(86) PCT No.: PCT/GB03/04725

§ 371 Date: Nov. 18, 2005

(30) Foreign Application Priority Data
Nov. 4, 2002 (GB) 0225676.6

(51) Int. Cl. A61K 31/353

(52) U.S. Cl. 514/454

(57) ABSTRACT

The invention relates to the use of one or more cannabinoids in the treatment of neuropathic or chronic pain.

### FIELD OF THE INVENTION

The invention relates to the use of one or more cannabinoids in the treatment of neuropathic or chronic pain.

### BACKGROUND TO THE INVENTION

The brachial plexus is formed from a group of combining spinal nerves that eventually divide to form the entire motor and sensory supply to the upper limb. Trauma to these nerves is associated with paralysis, loss of sensation and frequently chronic pain. Initial treatment is to repair the nerve damage through surgery. Although this surgery is often successful in restoring motor function, patients are often left with long term pain. The few studies which have reviewed pain following brachial plexus injury have shown that this pain is particularly difficult to treat.¹ Drugs are available to treat the pain but they have limited efficacy and are often associated with side effects. Improved treatments are urgently needed for this patient group.

Cannabis plants (Cannabis sativa) contain over 60 different cannabinoids². In the UK until 1971, British Doctors could prescribe oral tinctures of cannabis.³ Subsequently, cannabis and cannabinoids were placed in Schedule 1 of the Misuse of Drugs Act 1971 and for the past four decades, cannabis has been associated with illicit recreational use, largely by smoking dried plant material or resin from the flower heads to obtain a rapid absorption from the lung, giving a euphoric state or ‘high’. The principal psychoactive component in cannabis preparations is considered to be the cannabinoid Δ⁹ tetrahydrocannabinol (THC).

Cannabinoids affect almost every body system and like any other drug may have side effects.⁴ These are not usually severe and compare favourably with many other drugs with similar therapeutic targets for example; tricyclic antidepressants, phenothiazines, opioid and non-opioid analgesics and anticonvulsants. It has been estimated, based on extrapolation from mouse to man, that the lethal dose to effective dose (LD/ED) ratio is about 40,000 to 1.⁸ There have been no recorded deaths directly attributable to cannabis alone whereas in the UK approximately 600 people die each year following gastrointestinal haemorrhage, largely associated with NSAID use.⁷

Known pharmacological effects of cannabinoids⁴ include psychological effects, effects on perception, cognition, psychomotor performance and motor function, and analgesic, anti-emetic and sedative effects. Cannabinoids are known to cause a decrease in intra-ocular pressure and an increase in appetite. There are also cardiovascular effects; tachycardia and increased cerebral blood flow (with acute use), bradycardia and decreased cerebral blood flow (with chronic use), vasodilation and increased cardiac output. Effects on the respiratory system include bronchodilatation, airways obstuction (from smoking), and effects on ventilation. Aggravation of psychosis may occur in patients with schizophrenia.

Whether the recreational use of cannabis encourages escalation of dosage and progression to other dependency-producing drugs remains debatable. Many other therapeutic drugs have an abuse potential that might be considered to be more harmful and less reversible such as; benzodiazepines and opioids. However, experience with patients receiving opioids for pain relief shows that therapeutic use rarely leads to misuse,^{8,9} and the same is likely to apply to cannabinoids. Withdrawal symptoms from cannabinoid use are said to be short-lived (a few days) and mild in normal experimental subjects.³ Experience with the clinical use of Nabilone indicates that this is probably a minor and occasional problem. Psychological dependency definitely occurs in a small minority of recreational users. Some workers take the view that there is also a modest physical withdrawal syndrome when heavy users abruptly abstain,⁴ though this seems to be limited to a few nights of sleep disturbance and somatic anxiety symptoms.

A body of anecdotal evidence has emerged that suggests that patients with a range of conditions and, diseases can obtain significant symptom relief from illicit or ‘street’ cannabis. The evidence base includes reports from patients with rheumatoid arthritis, neuropathic pain, cancer pain and multiple sclerosis (MS).⁶ Cannabis use has tended to happen amongst patients with severe or intolerable symptoms that conventional therapies had failed to relieve and who had tried cannabis as a last resort.

Subsequently, interest has grown and research has been conducted into the therapeutic uses of cannabinoids.² This research has not been part of a coordinated programme and has involved small trials, often in single indications and has focussed mainly on purified oral formulations of the main psychoactive component, THC. One placebo-controlled study of oral THC in nine patients showed a statistically significant reduction in spasticity compared with placebo.¹² A second placebo controlled study of oral THC in 13 patients reported significant subjective improvements in spasticity.¹³ In many other cases however, the results have been inconclusive, but benefits have been evident even in small trials. These contradictory results are probably because routes of administration involving the gastro-intestinal tract (oral, rectal) are slow and produce variable effects, due to the poor and varied absorption from the gut. In these settings it has been difficult to titrate cannabinoids accurately to a therapeutic effect.

Currently the synthetic cannabinoid Nabilone® is the only cannabinoid preparation with a licence for medicinal use in the UK. Nabilone® capsules are indicated for intractable nausea and vomiting associated with cytotoxic chemotherapy. There has been insufficient evidence to secure regulatory approval in any other indications. Purification of a single cannabinoid may be a contributory factor in limiting efficacy in therapeutic areas where strong anecdotal evidence has suggested a therapeutic benefit from smoked cannabis. A mixture of many cannabinoids is delivered when cannabis is smoked,² but smoking clearly is an inappropriate delivery system for a medicinal product. The smoke is inconsistent in composition and contains potential carcinogens from incomplete combustion, similar to tobacco smoke.⁴

The findings of the House of Lords Select Committee on Science and Technology recommended that clinical trials of cannabis for the treatment of MS and chronic pain be mounted as a matter of urgency and that research should be promoted into alternative modes of administration which would retain the benefits of the rapid absorption offered by smoking without the associated adverse effects (Section 28, References, 1). The Institute of Medicine report on medicinal cannabis also recommended that therapeutic trials be undertaken on non smoked forms of cannabis-based medicines.¹¹

The applicant has developed cannabis based medicinal extracts (CBME), from whole cannabis plants as disclosed in WO 02/064109. The extracts are derived from strains of plants developed to produce high and reproducible yields of specified cannabinoids. The extracts from these plants contain a defined amount of the major cannabinoid, plus trace amounts of minor cannabinoids. The major cannabinoids constitute not less than 90% of the total cannabinoid content of the extracts. It is thought that the minor cannabinoids may add to the overall therapeutic profile of the CBMEs and may play a role in stabilising the major components. Currently, two CBME preparations have reached phase 3 clinical studies, “THC” in which Δ⁹ tetrahydrocannabinol is the major cannabinoid, and “THC:CBD 1:1”, containing substantially equal proportions of THC and cannabidiol (CBD) as the major cannabinoids.

Sublingual and inhaled CBME preparations have been developed, to achieve rapid absorption of the type seen from smoking cannabis, and minimise absorption by the oral route, which is subject to first pass metabolism. Evidence collected from pooled phase 2 “n of 1” studies (single case within patient crossover studies)^{5,10} has indicated that these routes of administration are not associated with the titration problems of the oral route. CBME dosing is similar to patient controlled analgesia (PCA), most commonly used to deliver opioids for control of post-operative pain. Small increments are delivered each time patients require them, up to a maximum daily limit. The phase 2 “n of 1” data have helped to define the effective dose delivered per actuation, and the recommended maximum doses. The data also indicate that the therapeutic benefits of CBMEs are delivered at doses below those which cause a sensation of a ‘high’, and that onset of the ‘high’ may be an indicator of overtitration.

A large proportion of the MS patients who reported peripheral pain as part of their symptomatology in the “n of 1” studies showed marked improvement in pain scores with CBME therapy, usually with few persisting side-effects once the optimum dose had been reached.¹⁰ Peripheral MS pain may have a neuropathic element but is often multifactorial. Pain of purely neuropathic origin in MS is difficult to diagnose clinically. Studies were set up to investigate and study the efficacy of CBME in relieving neuropathic pain and chronic pain following brachial plexus injury. In this condition, chronic pain is attributed to nerve injury, that is, it is neuropathic in origin.

Of primary interest in this study is the efficacy of CBME in relieving neuropathic pain, in comparison to placebo. Brachial plexus injuries may follow stretching caused by shoulder dislocation (dystocia), breach extraction or hyper abduction of the neck in abnormal presentations during labour. The injuries can be due to simple stretching, haemorrhage within a nerve, tearing of the nerve or root or avulsion of the roots with associated cervical cord injury. The injuries may also be due to trauma of the clavicle or humerus or subluxation of the shoulder or cervical spine. There are a number of other conditions such as ERB's Palsy (upper brachial plexus injury) (lower plexus injury). All of these conditions are examples of neuropathic pain and are responsible for considerable morbidity. The prognosis for recovery in any of these conditions is poor and the pain associated with them is excruciating. To date there is very little than can be done to relieve pain in patients with these conditions.

Surprisingly, it has been found that extracts of cannabis containing as principal cannabinoids Δ⁹-tetrahydrocannabinol (THC) and cannabidiol (CBD) in differing proportions are not only effective, but are so at low doses. In this regard the applicant has previously determined that in order to treat pain in Multiple Sclorosis patients it has typically been necessary to provide a daily dose in the range of 30-50 mg. Thus, the term low dose as used herein refers to a mean daily dose of less than 40 mg.

### DESCRIPTION OF THE INVENTION

According to a first aspect of the present invention there is provided the use of one or more cannabinoids in the manufacture of a medicament for use in the treatment of neuropathic or chronic pain.

In one embodiment the medicament is provded in a form capable of delivering a mean daily dose of less than 37.5 mg.

Where the medicament is packaged for delivery as e.g. a sub lingual or buccal spray a typical mean daily dose will be less than 25 mg, and typically in the range 5-25 mg.

Prefered cannabinoids are THC and/or CBD, more preferably in the form of a CBME.

According to a second aspect of the present invention there is provided the use of one or more cannabinoids in the manufacture of a medicament for use in the treatment of sleep disturbance.

The invention is further described, by way of example only, with reference to the following Examples and Figs in which:

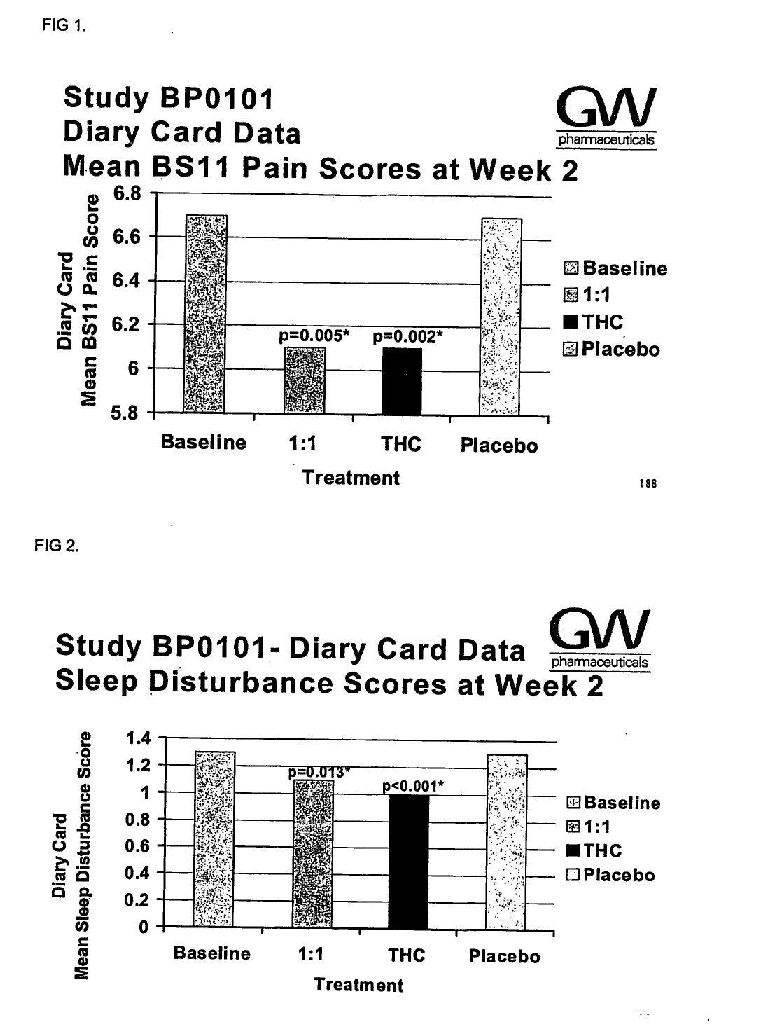

FIG. 1 shows diary card data for treatments with a high THC or THC/CBD CBME. Pain Score is compared to baseline and placebo;

FIG. 2 shows diary card data showing sleep disturbance scores (change from baseline) for treatments with a high THC or THC/CBD CBME;

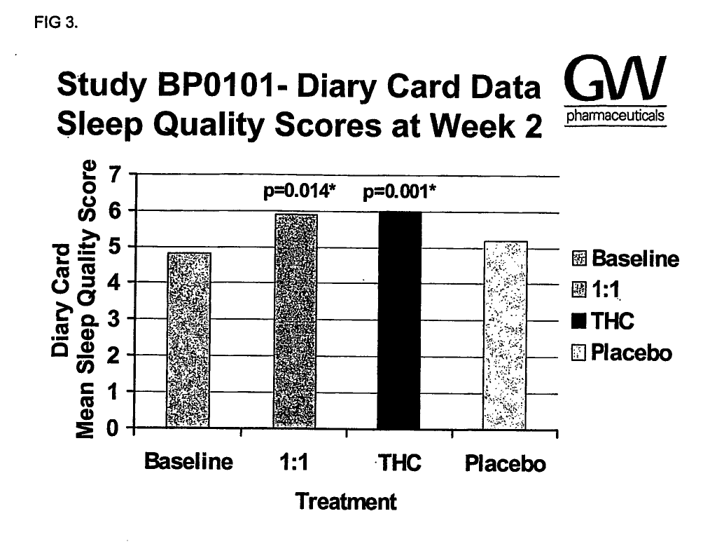

FIG. 3 shows diary card data showing sleep disturbance scores for treatments with a high THC or THC/CBD CBME. Sleep disturbance is compared to baseline and placebo.

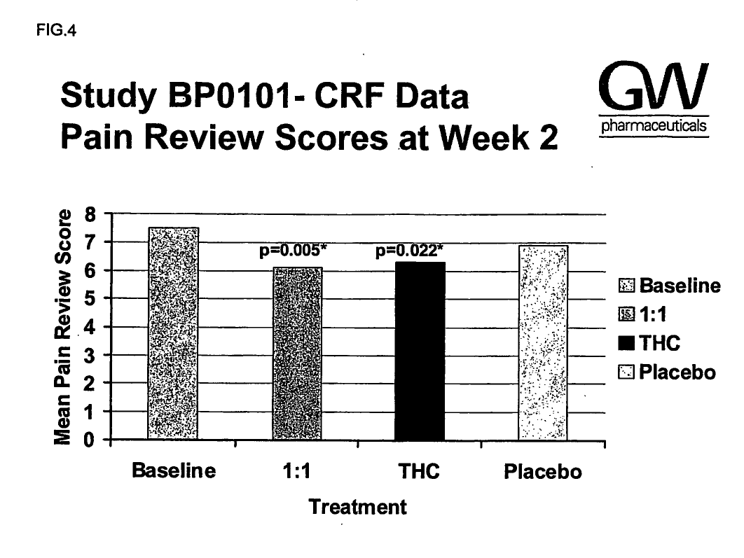

FIG. 4 shows pain review scores treatments with a high THC or THC/CBD CBME.

In clinical trials of cannabis extracts, fractional doses of 2.5 mg were given to patients with conditions such as the pain of multiple sclerosis to achieve a total daily dose of approximately 40-50 mg of THC or this dose of THC combined with an equal quantity of CBD. In refractory condition such as brachial plexus avulsion (BPA) it might be expected that higher doses would be required which would take the total daily dose into the range where cognitive impairment was produced in patients. Surprisingly it was found that patients with BPA who were treated with the same preparations obtained significant relief at doses of approximately one half of this level. It was also noted that objective measurements of pain (box scale 11—a validated pain score) showed that THC and CBD produced statistically significant reductions in BS11 pain score and both were highly significantly different from placebo and base line scores. Sleep disturbance was also reduced by THC, and THC in combination with CBD, and there was also an improvement in sleep quality at week 2. Both THC and CBD produced statistically highly significant improvement.

### EXAMPLE 1

A clinical trial was carried out in 48 patients with chronic pain due to brachial plexus injury. This was a double blind, randomised, three-way crossover study comparing two different sublingual cannabis-based medicine extracts (CBMEs) with placebo. The active treatments were given in the form of a sublingual spray. Each spray contained 2.5 mg of THC or 2.5 mg of THC plus 2.5 mg of CBD in the form of an alcoholic solution of a cannabis extract. The patients titrated the dose up to the level at which pain relief was obtained; assessments were made by the patient diary scores and by the clinicians and nursing staff.

FIG. 1 shows the diary card scores based on the box score 11 (BS11) in comparisons with placebo. The mean number of sprays for the patients receiving THC was 7.26 and for the 1:1 ratio THC:CBD was 6.93, compared with 7.15 for placebo. These doses correspond to total daily doses of approximately 18 mg for THC and 17 mg when THC was given in conjunction with CBD.

A further surprising finding was that the number of sleep disturbances in these patients (who have their sleep duration and quality frequently disturbed) obtained improvement in this parameter. FIGS. 2 and 3 show that sleep disturbance scores at week 2 were highly significantly statistically improved both with THC and the THC:CBD combination compared with placebo.

FIG. 4 shows pain review scores showing the effect of THC and THC:CBD in 1:1 ratio at week 2. The differences from placebo was highly significantly statistically, not only by diary card but by mean pain review score (clinician assessment).

Significantly the relief of pain in these patients from the cannabis extract was achieved at doses which did not cause significant cognitive impairment.

### COMPARATIVE EXAMPLE

In previous studies, which examined the effect of cannabis-based medicine on pain relief in multiple sclerosis, pain relief was typically achieved in the range 25-50 mg/day approximately.

It is therefore surprising that in patients with neuropathic pain, particularly brachial plexus pain, which is notoriously difficult to treat, relief was obtained at lower doses.

### REFERENCES

1. Berman J, Birch R, Anand P. Pain following human brachial plexus injury with spinal cord root avulsion and the effect of surgery. Pain 1997; 75:199-207.
2. Mechoulam R. Cannabinoid chemistry. In: Marijuana—chemistry, pharmacology, metabolism and clinical effects. Mechoulam R ed. Academic Press, London. 1973:1-99.
3. Therapeutic uses of cannabis. British Medical Association. 1997. 1-6. Harwood Academic Publishers. Morgan DR ed.
4. Ashton CH. Adverse effects of cannabis and cannabinoids. Br J Anaesth 1999; 83:637-49.
5. Investigator Brochure—Cannabis based medicine extract sublingual formulations. GW Pharmaceuticals plc, February 2001, Edition 5.
6. Grinspoon L, Bakalar J B. Marijuana, the forbidden medicine. 1993. Yale University Press, 138.
7. Prescribers Journal 1998; 38.4:213.
8. Porter J, Jick H. Addiction rare in patients treated with narcotics. N Engl J Med 1980; 802:123.
9. Portenoy R K. Chronic opioid therapy in non-malignant pain. J Pain Symptom Manage 1990; 5 (Suppl 1):46-62.
10. Unpublished data. GW Pharmaceuticals plc.
11. House of Lords Select Committee on Science and Technology: Cannabis: The scientific and medical evidence. 1998; 9^{th} Report.
12. Petro D J, Ellenberger C. Treatment of human spasticity with Δ⁹ tetrahydrocannabinol. J Clin Pharmacol 1981; 21:413S416S.
13. Ungerleider J T, Andrysiak T. Fairbanks L et al. Delta-9-THC in the treatment of spasticity associated with multiple sclerosis. Adv Alc Subs Abuse 1988; 7:39-50.

1. A method of treating brachial plexus avulsion in a human patient comprising administering to a patient in need thereof an effective amount one or more cannabinoids.

2.-9. (canceled)

10. The method as claimed in claim 1 which additionally involves the treatment of neuropathic pain caused by brachial plexus avulsion.

11. The method as claimed in claim 1 which additionally involves the treatment of sleep disturbance caused by brachial plexus avulsion.

12. The method as claimed in claim 1, wherein the one or more cannabinoids comprise delta-9-tetrahydrocannabinol (THC).

13. The method as claimed in claim 1, wherein the one or more cannabinoids comprise cannabidiol (CBD).

14. The method as claimed in claim 1, wherein the one or more cannabinoids comprise both THC and CBD.

15. The method as claimed in claim 14, wherein the THC and CBD are provided in a ratio of between 2:1 and 1:2.

16. The method as claimed in claim 15, wherein the THC and CBD are provided in a ratio of substantially 1:1.

17. The method as claimed in claim 12, wherein the medicament is provided in a form capable of delivering a mean daily dose of less than 37.5 mg THC.

18. The method as claimed in claim 12, wherein the medicament is packaged for delivery as a sublingual or buccal spray to provide a daily dose of less than 25 mg THC.

19. The method as claimed in claim 1, wherein the cannabinoids are present as a cannabis based medicinal extract (CBME).

20. (canceled)
